(19) United States
(12) Patent Application Publication
(10) Pub. No.: US 2025/0172575 A1
(43) Pub. Date: May 29, 2025

(54) QUANTITATIVE BLOOD COLLECTION VIAL AND SYSTEM AND METHOD FOR DETERMINING HEMATOCRIT

(71) Applicant: DAXOR CORP., Oak Ridge, TN (US)

(72) Inventors: Jonathan Feldschuh, Jackson Heights, NY (US); Adam Michael Cable, Maryville, TN (US); Jackson Rieb, Maynardville, TN (US); Linda J. Cooper, Maryville, TN (US)

(73) Assignee: DAXOR CORP., Oak Ridge, TN (US)

(21) Appl. No.: 18/958,223

(22) Filed: Nov. 25, 2024

Related U.S. Application Data
(60) Provisional application No. 63/602,727, filed on Nov. 27, 2023.

(51) Int. Cl. G01N 33/80; A61B 5/15; G01N 33/58; G01N 33/60; G01N 35/00

(52) U.S. Cl. CPC G01N 33/80; A61B 5/1433; G01N 33/582; G01N 33/60; G01N 35/00732; G01N 2035/0091

(57) ABSTRACT

A device is presented for storing, transporting, measuring, and collecting blood, having the properties of a precisely determined volume, and a geometry suitable for the entire device to be efficiently placed in the counting chamber of a detector, such as the counting well of a gamma counter. Also presented are a system and method for determining the Hematocrit (Hct) of a subject.

### CROSS-REFERENCE TO RELATED APPLICATION

This application claims the benefit of U.S. Provisional Patent Application No. 63/602,727, filed on Nov. 27, 2023, the contents of which are herein incorporated by reference in their entirety into the present application.

### FIELD OF THE INVENTION (TECHNICAL FIELD)

The present invention relates to systems and methods for storing, transporting, measuring, collecting, and analyzing blood.

### BACKGROUND

Blood is a complex fluid, comprising a watery plasma containing various proteins (principally albumin) and large cells (principally red blood cells). Quantitative collection of blood must deal with a variety of factors specific to blood including: the potential for clotting (especially when exposed to air); the potential for hemolysis (when the fluid is disturbed or agitated excessively); the potential for settling (the tendency of red blood cells to sink when blood is allowed to stand under the influence of gravity, resulting in an inhomogeneous mixture of red cells and plasma in the sample); the potential for surface adhesion (the proteins in blood are generally “sticky” and will adhere to most plastic surfaces, and will resist displacement under typical flow conditions); and the potential for non-laminar flow caused by partial separation on the blood components caused by the geometry of the blood pathway through the vial. As a result of these issues, quantitative measurements of blood that require volumes greater than 0.15 ml generally involve a cumbersome, multi-step process. First blood is collected (e.g. into a vacutainer tube at the bedside) and then it must be transferred quantitatively into a separate container (e.g. using a precision pipette at a laboratory bench). Pipetting of whole blood is quite difficult and potentially inaccurate, because of all the issues including settling, adhesion, hemolysis, etc. Centrifuging whole blood to get access to plasma is feasible, but this adds time and complexity to a quantitative process, and still requires skill in pipetting to ensure accuracy (e.g. to pipette only plasma and no red cells). For all these reasons a solution enabling direct collection of precise amounts of whole blood directly from a patient for the purposes of quantitative measurement is desirable.

Hct is defined as the volume percentage of red blood cells in whole blood. Hct is typically measured in a variety of ways. Automated analyzers performing Complete Blood Count (CBC) testing typically measure the Hct as the product of Mean Cell Volume (MCV) and the Red Blood Cell (RBC) Count, with RBC in units of concentration. These CBC devices are large, complex, expensive, and laboratory-based. The gold standard method for direct determination of Hct uses the percentage of volume method, by which the volume of spun red cells is directly compared to the volume of the original sample. Typical embodiments of this method use capillary tubes which are filled with blood, then spun in a microcentrifuge; the proportion of red cells can be measured by comparing linear distance of the colored portions of the capillary tube. Various Point-of-Care (POC) devices exist which can measure Hct—or Hemoglobin (Hb) concentration, which can be converted to Hct via a numerical factor-but these devices generally lack the precision and accuracy, and/or the extended range of the lab methods. In the present invention, a single tracer is used that marks one component of the whole blood (either the red cells or the plasma), and that tracer is then measured in samples of whole blood and extracted plasma.

Many forms of quantitative vials for fluid collection exist. These are generally designed to collect very small quantities of fluid, which are then measured by some means. The present invention deals with the situation where the vial is designed to be fillable using an ordinary syringe without any special training; and where the volume of fluid to be collected is at least 0.5 ml (i.e. greater than the approximate maximum of 0.15 ml using capillary action). The use of a precise geometry for the vial facilitates applications where measurements of a sample (collected or monitored in the vial) are compared with measurements of a reference standard (contained in an identical vial, or in a vial with a known ratio of volume to the quantitative vial). An example of such an application would be the measurement of an unknown volume using the indicator dilution method, whereby a known amount of tracer is introduced into the unknown volume; the amount of tracer present in a sample collected from the unknown volume can be directly compared to the amount of tracer present in a reference standard created by diluting the same tracer into a known volume, and filling an identical vial with the resulting dilution.

### SUMMARY OF THE INVENTION

A device is presented for storing, transporting, measuring, and collecting blood, having the properties of being easily filled to a precisely determined volume, and a geometry suitable for the entire device to be efficiently placed in the counting chamber of a detector (such as the counting well of a gamma counter). This device features in a method for determining Hematocrit in a sample of blood, using a tracer which is counted in precise volumes of the blood sample, and in plasma derived from the same sample.

### BRIEF DESCRIPTION OF THE SEVERAL VIEWS OF THE DRAWINGS

FIGS. 1A-1F show various exploded views of one embodiment of a quantitative vial.

FIG. 2 shows one embodiment of an assembly process for a quantitative vial.

FIGS. 3A-3G show various views of one embodiment of the bottom section of the quantitative vial.

FIG. 4 shows a vial being filled using a syringe.

FIGS. 5A-5C show a method of filling a vial and controlling the pressure applied so that it does not exceed the WEP of the vial, thus protecting the membrane from potential failure.

FIGS. 6A-6B show an embodiment where a quantitative vial is counted in a small detector apparatus with a touchscreen user interface.

FIGS. 7A-7D show embodiments of quantitative vials that have different shapes.

FIG. 8 shows one embodiment of a method of determining Hct using a tracer.

FIGS. 9A-9D show an embodiment of a method of determining Hct using a tracer, using quantitative vials which can be filled to a precise volume with a syringe via luer-lock access, and a plasma separation chamber.

### DETAILED DESCRIPTION OF THE INVENTION

The invention provides a vial of precise volume, comprising an ingress with a standard fitting for accepting fluid and a valve to prevent backflow or outflow of the fluid, and an egress incorporating a membrane which passes air but retains fluid. In one embodiment, the standard fitting is a luer-lock mechanism.

In a one embodiment, the vial is formed from four separate components: an ingress adapter incorporating a luer lock and a valve, a membrane with pressure resistance, defined as water entry pressure or WEP, sufficient to prevent flow of fluid, a lid to which the membrane is affixed, and a vial body which connects the lid to the adapter, defining a precise volume. In one embodiment, the components are assembling using adhesive (such as cyanoacrylate). In a preferred embodiment, the components are assembled using injection molding.

In one embodiment, the vial is designed to be compact (i.e. having a small height relative to the depth of a counting well), such that the entire sample in the vial is deep within the well. This increases the counting efficiency for the sample and minimizes counting geometry effects for inhomogeneities in the sample. In one embodiment, the well is a complete cylindrical cutout, allowing vials to be inserted from one side and removed from the other.

In one embodiment, the membrane is supported by a dense structure, increasing its effective WEP rating. In another embodiment, the membrane is supported by a sparse structure, increasing its effective surface area for passage of air.

In one embodiment, the vial body is formed from transparent material with sufficient clarity to allow visual inspection of the vial by the user, to verify that the vial is completely filled, with no visible air spaces. A partial list of materials that are both injection moldable and have sufficient clarity includes: Acrylic (PMMA), Acrylonitrile butadiene styrene (ABS), Nylon (polyamide, PA), Polycarbonate (PC), Polyethylene (PE), Polyoxymethylene (POM) (Delrin), Polypropylene (PP), Polystyrene (PS), Thermoplastic Elastomer (TPE), and Thermoplastic Polyurethane (TPU). Sufficient clarity also allows for quantitative measurement of light emitted from or absorbed by the liquid in the vial when placed in a suitable detection apparatus.

In one embodiment, the vial incorporates smooth curved transitions between all components to ensure that no air is trapped in the vial while it is filled by eliminating spaces where are bubbles could form and thereby prevent the exact desired volume from being achieved in the vial.

In one embodiment, the vial is fitted with an additional access point, equipped with an adapter for accepting liquid via a luer lock or standard threaded connection and a valve, to provide a means of applying or relieving pressure. Such an adapter with a suitable pressure release valve can ensure that the vial membrane does not experience pressure that exceeds the membrane WEP, thus preventing leakage or failure of the membrane.

In one embodiment, the inner surfaces of the vial are treated, either before or after assembly, with a finish coating that has the property of having low adhesion for blood. A partial list of methods of achieving such coatings include the use of super-hydrophobic polystyrene coatings such as MnO2/PS or ZnO/PS, silica nano-coating, mold temperature hydrophobicity tuning, or zwitterion coating.

In one embodiment, the vial can be shaped to fit the geometry of a detector. For example, a cylindrical vial could fit completely in a cylindrical well such as is typically found in a gamma counter. A conical vial, alternatively, concentrates the sample in the bottom of such cylindrical well, minimizing variations in counting caused by settling effects in the vial by concentrating most of the sample at the bottom of the vial. A spherical or rectangular prism shape for the vial can be shaped to fit a spherical or rectangular prism well. In one embodiment, the detector well has a fold-over design allowing the vial to be partially or completely encased in the detector.

In another preferred embodiment, an injectable tracer and dimensionally identical reference standard and collection vials are included as components of a kit to enable an indicator dilution measurement to be performed. A reference standard vial is prepared by diluting tracer from the same production lot as the injectable tracer) into a known volume (e.g. 1000 ml).

In one embodiment, a separate disposable sleeve is provided, of suitable size and dimensions to accept the vial, with the vial-enclosed sleeve fitting into the detector. The sleeve serves to facilitate handling of the sample vial, and to protect both the user and the detector from possible contamination by the blood contained within the vial, as might occur if the membrane were to fail or be damaged, or the cartridge were otherwise to leak. The sleeve also facilitates agitation of the sample, whereby the sample vial is shaken by hand to counteract any settling that may have occurred in the time between filling the vial and counting it in the detector.

The invention also provides a method for determining Hct where a single quantitative tracer that labels the plasma is introduced into a sample of whole blood. This introduction can take place after the blood is collected from the subject, or the tracer can be injected into the subject and allowed to equilibrate in the subject. A precise aliquot of the whole blood is counted to determine the quantity of tracer in whole blood, and a precise aliquot of plasma derived from the whole blood is counted to determine the quantity of tracer in the plasma. These counts are then used to derive the Hct using the equation

$$Hct = 1 - \frac{{A}_{wb}}{{A}_{p}} ,$$

where A_{wb} is the activity of the tracer in the whole blood sample, and Aₚ is the activity of the tracer in the plasma sample. In one embodiment, the method comprises
a. introducing a tracer into the blood of the subject that marks the plasma component of the blood, and allowing sufficient time for said tracer to become equally distributed throughout the blood of the subject;
b. collecting a sample of the subject's blood;
c. measuring A_{wb}, the activity of the tracer in the whole blood sample from step (b);
d. extracting plasma from the whole blood sample from step (b);
e. measuring Aₚ, the activity of the tracer in the plasma sample from step (d); and
f. calculating the Hct of the subject using the equation

$$Hct = 1 - \frac{{A}_{wb}}{{A}_{p}} .$$

In one embodiment, the tracer is a radionuclide such as I-131, I-125, or Tc-99m bound to a plasma protein such as human serum albumin. In another preferred embodiment, the tracer is a radionuclide such as Tc-99m bound to red cells.

In one embodiment, the tracer is a fluorophore such as ICG, Fluorescein, or IRDye CW800 that is detectable directly in plasma, or a fluorophore bound to a plasma protein.

In one embodiment, quantitative collection vials are used to contain precise aliquots of plasma and blood, enabling POC use by eliminating the need for laboratory procedures such as precision pipetting.

In one embodiment, a plasma separation membrane is used to separate plasma from whole blood, enabling POC use by eliminating the need for laboratory procedures such as centrifugation. A plasma separation chamber has a plasma separation membrane blocking the egress, such that whole blood is introduced to the chamber at the ingress, and syringe pressure is sufficient to produce plasma at the egress.

In one embodiment, an automated POC blood volume analyzer calculates blood volume information for a subject determined by the injection of a plasma tracer and the application of the volume dilution method; samples are taken from the subject and placed in quantitative counting vials and counted at the bedside in a portable counter; plasma is extracted from one or more of the post-injection whole blood samples using a plasma separation membrane, such as a Polysulfone (PES) membrane.

The invention also provides a system for determining the Hematocrit (Hct) of a subject, consisting of a tracer that marks the plasma component of the blood, a concentration counter capable of measuring the activity of said tracer, containers for presenting samples to the concentration counter, and a plasma separator capable of producing a sample of plasma from a sample of whole blood, using the methods disclosed herein.

In one embodiment, the containers for presenting samples to the concentration counter are vials that are filled to a precise volume, V_{wb} and Vₚ for whole blood and plasma, using an ordinary syringe, wherein the activities are calculated as

$${A}_{wb} = \frac{{Counts}_{wb}}{{V}_{wb}} and {A}_{p} = \frac{{Counts}_{p}}{{V}_{p}} ,$$

and wherein the vials are vials of precise volume, comprising an ingress with a standard fitting for accepting fluid and a valve to prevent backflow or outflow of the fluid, and an egress incorporating a membrane which passes air but retains fluid.

In one embodiment, the plasma separator is a chamber with an input port for whole blood, a plasma separating membrane, and an output port that connects to the ingress of the vials.

The invention further provides a system for automatically analyzing blood of a living subject at the point of care (POC), comprising a concentration counter configured to analyze one or more samples, a user interface operatively connected to the concentration counter and configured for entry and display of information, one or more processors operatively coupled to a memory and configured to execute programmed instructions stored in the memory to carry out a method comprising the steps of:
measuring a concentration of a tracer in a vial containing whole blood from the subject in the concentration counter to determine a background count of said tracer in the subject's blood;
recording the time at which the subject is injected with a precise, known volume of said tracer;
counting in the concentration counter blood drawn from the subject in one or more additional vials at one or more timed intervals after the injection;
determining the subject's Hct using plasma obtained from the whole blood samples;
calculating, by the one or more processors, a blood volume (BV), plasma volume (PV), and red cell volume (RCV) for the subject using the subject's Hct;
calculating, by the one or more processors, an ideal blood volume (iBV), ideal plasma volume (iPV), and ideal red cell volume (iRCV) for the subject patient based on the subject's height, weight, and gender; and
displaying, by the one or more processors, at the user interface, the calculated volumes.

The essential requirements for a quantitative blood collection vial are that it enable collection of a precise amount of blood directly from a subject; that the amount of blood is not limited to the quantity that can be collected by capillary action; that the collection process not require specialized skills beyond those possessed by a phlebotomist; and that the collected sample can immediately be counted in a quantitative detector in the collection vial.

Gamma scintillation detectors have a geometry effect. When a sample is placed into a counting well, the sample is surrounded on nearly all sides by the detector crystal, with the unavoidable exception of the solid angle subtended by the well opening. The farther down the well, the more efficient the counting will be; in the extreme case, a sample placed at the very opening of the well will have approximately half the counting efficiency, ignoring effects of absorbance by the well liner. Therefore, it is desirable to concentrate as much of the sample at the bottom of the well (i.e. at the bottom of the vial). Several embodiments are presented that achieve this objective.

A kit is provided for performance of an indicator dilution measurement, consisting of a plurality of labelled dimensionally identical vials as disclosed herein provided together in a suitable package. The kit also includes a radioactive tracer in a ready-to-inject container and a standard with a known dilution of said tracer in a vial that has a known ratio of volume to the vials.

A system is provided for performing an indicator dilution measurement using said kit to determine an unknown volume whereby an injectable tracer is injected into an unknown volume. After a short interval (to ensure mixing has occurred), a sample is collected from the subject into an empty collection volume of identical dimensions. By counting the standard vial and the patient vial, the patient volume can be computed using the simple ratio:

$$patient . volume = ( \frac{standard . counts}{patient . counts} ) standard . volume . \qquad ( 1 )$$

One skilled in the art will recognize how background measurements (from patient and room) can be accounted for by subtracting the relevant counts, and how multiple time samples can be taken to improve the accuracy of the measurement and account for loss of the tracer from the circulation over time.

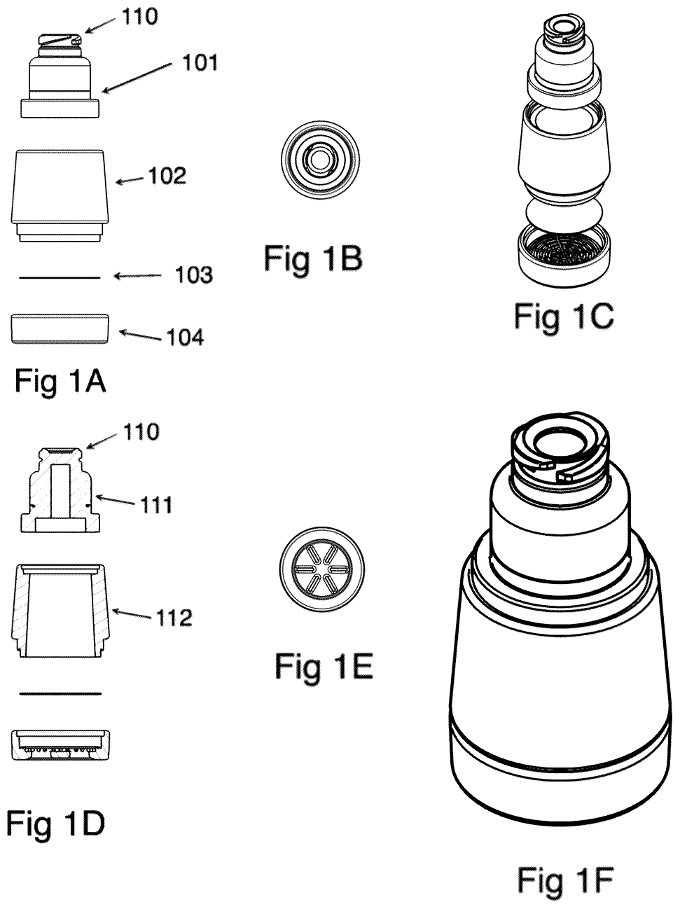

FIGS. 1A-1E shows various exploded views of one embodiment of a quantitative vial, respectively side (1A), above (1B), oblique (1C), cutaway side (1D), and below (1E). The vial is fashioned from four components: a luer-lock fitted valve (101), a body cavity (102), a membrane that allows passage of air but blocks the passage of fluids below a certain WEP (103), and a base cap (104). The valve (101) features threads for a luer-lock mechanism (110), and a valve mechanism (111) which is open when the luer-lock connector is used. FIG. 1F shows (at larger scale) the assembled vial.

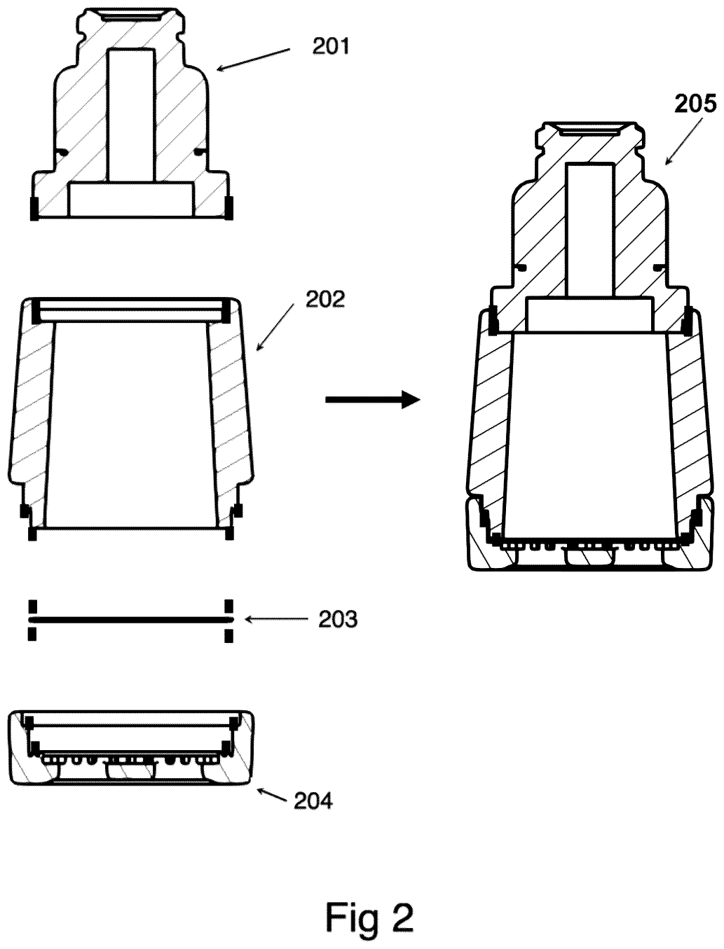

FIG. 2 shows one embodiment of an assembly process for a quantitative vial, using the application of adhesive. The solid black areas indicate the places on the four parts-a luer-lock fitted valve (201), a body cavity (202), a membrane (203), and a base cap (204)—to which a suitable adhesive such as cyanoacrylate is applied. Note that as this is a cross-section view, the adhesive would be applied around the circumference of each part at the indicated spot (denoted by solid black). After assembly of the parts, the finished vial (205) is shown. Alternatively, the black areas in 201-204 depict regions of the parts that would be subject to another adhesive process, such as ultrasonic welding, with certain areas of the plastic fusing during the ultrasonic application process to form the finished piece (205).

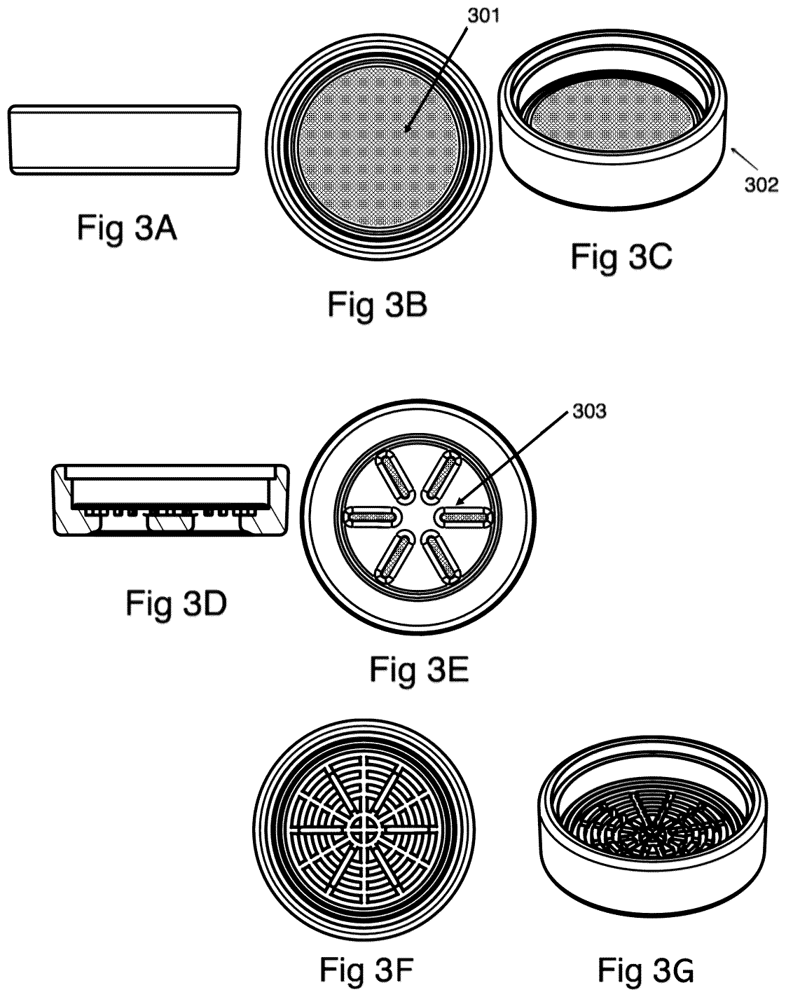

FIGS. 3A-3G show various views of one embodiment of the bottom section of the quantitative vial, respectively side (3A), above (3B), oblique (3C), cutaway side (3D), and below (3E). The membrane (301) is supported by the base cap (302), which includes a number of perforations (303) that allow air to exit through the membrane and out of the vial. One skilled in the art would recognize that many arrangements of perforations are possible, with dense material/minimal perforations optimizing support of the membrane, and sparse material/maximal perforations optimizing air flow through the membrane. Two additional views of the base cap (302) without the membrane (301) in place are shown from above (3F) and oblique (3G), depicting the support structure for the membrane that provides both air egress and physical support.

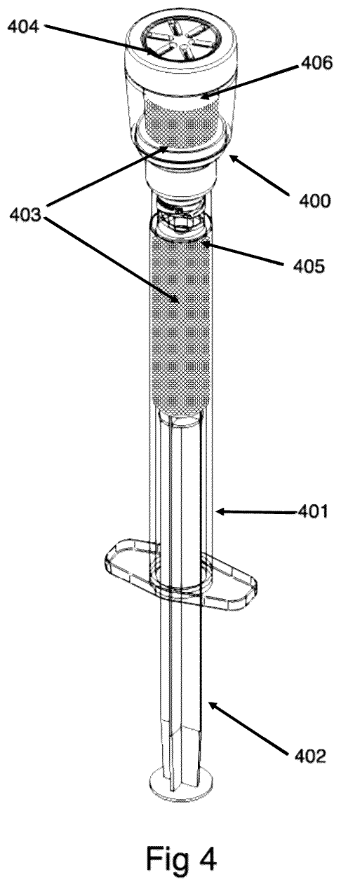

FIG. 4 shows a vial (400) being filled using a syringe (401), with the syringe and vial connected via the luer-lock connection (405). As the syringe plunger (402) is pressed into the body of the syringe, blood (403) passes from the syringe into the vial. The air space in the vial (406) is gradually eliminated, as air is forced out of the membrane-covered, perforated end of the vial (404). Complete, quantitative filling of the vial is signaled to the operator by a combination of signals: the disappearance of the air space (406), and a palpable increase in resistance to the plunger (402) as the air is eliminated and the fluid resists compression.

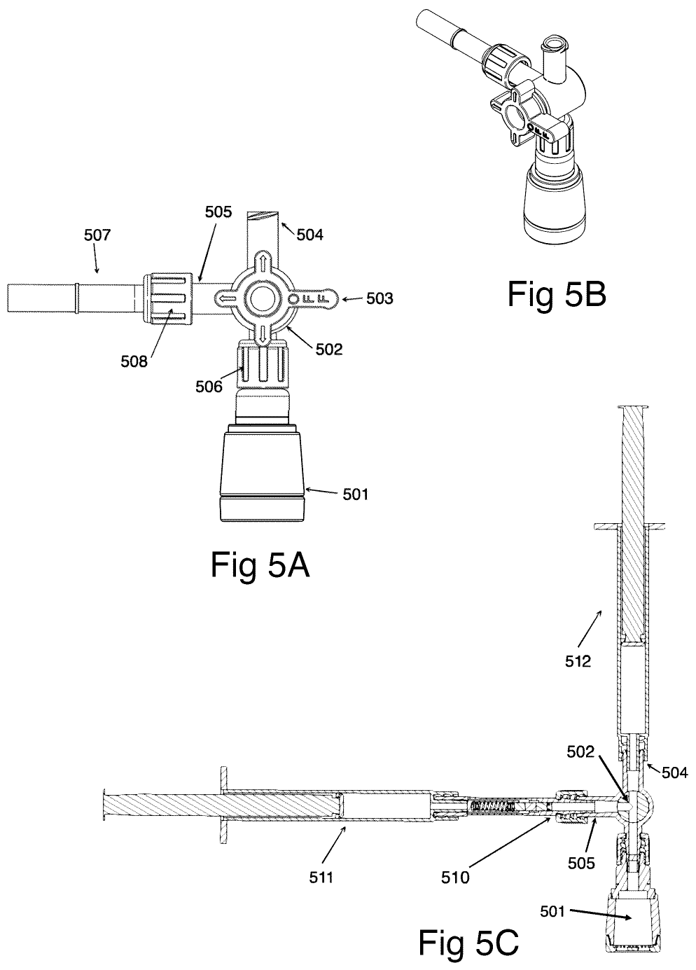

FIGS. 5A-5B show two views (side, 5A, and oblique, 5B) of a method of filling a vial (501) and controlling the pressure applied so that it does not exceed the WEP of the vial, thus protecting the membrane from potential failure. The vial (501) is connected to a 3-way stopcock (502) controlled by a directional switch (503); the stopcock has a female connecter (506) that connects to the vial (501), and two male connectors (504,505). A pressure gauge (507) with a female connector (508) is attached to one of the male connectors on the stopcock (505). A syringe with blood (not shown) can be attached to the other female connector (504). When the syringe plunger is depressed, the cartridge fills with blood (as shown in FIG. 4), with the pressure gauge (507) displaying the current pressure. The operator can control the force used on the plunger and monitor the pressure gauge during filling to ensure that the pressure readings stay below the membrane WEP threshold. Note that the switch (503) is shown in the position that allows the pressure gauge to be read during filling; if the switched were rotated 180 degrees (so that “OFF” pointed towards the pressure gauge) then the gauge would not be active, and the cartridge would be filled as in FIG. 4. One skilled in the art would recognize that many other means and arrangements exist for monitoring the pressure while filling the vial so as to avoid exceeding the WEP. FIG. 5C shows one such method, in which a pressure relief valve replaces the pressure gauge. In this configuration, the vial (501) is attached to the 3-way stopcock (502) as before, and the left-hand connector (505) of the stopcock is attached to a pressure relief valve (510), which in turn is connected to a waste reservoir, here shown as a syringe (511). When a fill syringe with blood (512) is attached to the top connector (504) of the stopcock, the pressure relief valve (510) will channel blood to the waste reservoir (511) when excessive filling pressure is applied, thus protecting the vial.

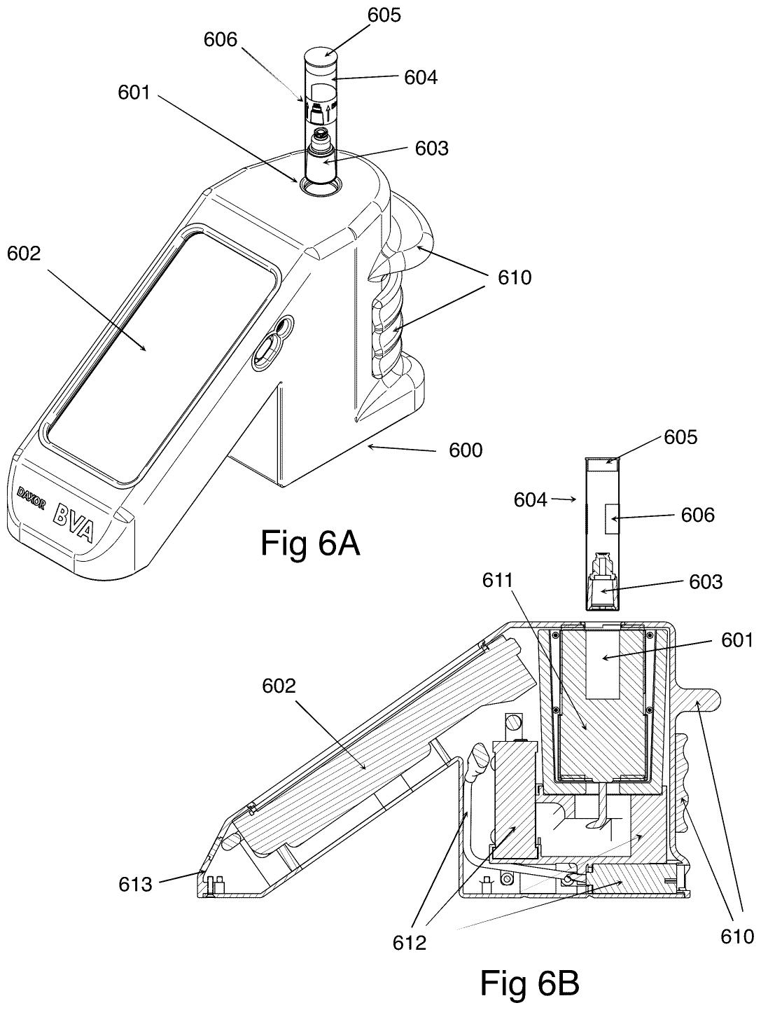

FIGS. 6A-6B show an embodiment where a quantitative vial (603) is counted in a small detector apparatus with user interface (600), shown in oblique (6A) and cross sectional (6B) views. The apparatus (600) is controlled by an embedded touchscreen device (602) and other associated electronic components (612), all contained within an enclosure (613) that includes ergonomic molded contours (610) that make the device easier to hold and balance, as the detector component (611) is the heaviest part of the apparatus. The vial (603) is first placed in a clear protective sleeve (604) with a cap (605), to protect the apparatus and user from any potential spills. The sleeve (604) has a label (606) which displays the correct orientation for the vial, as well as providing a surface for identification of the sample (e.g. patient I.D. or sample number or time). The sleeve also facilitates agitation of the blood in the vial, as the user can shake the sleeve up and down to counteract any settling of the blood that may have occurred. The sleeve (604) is inserted into the counting well (601), where the detector crystal (611) can detect and quantify radiation in the sample. Note that the sleeve (604) is longer than the well (601), which allows the user to easily remove the sleeve from the well when counting is complete.

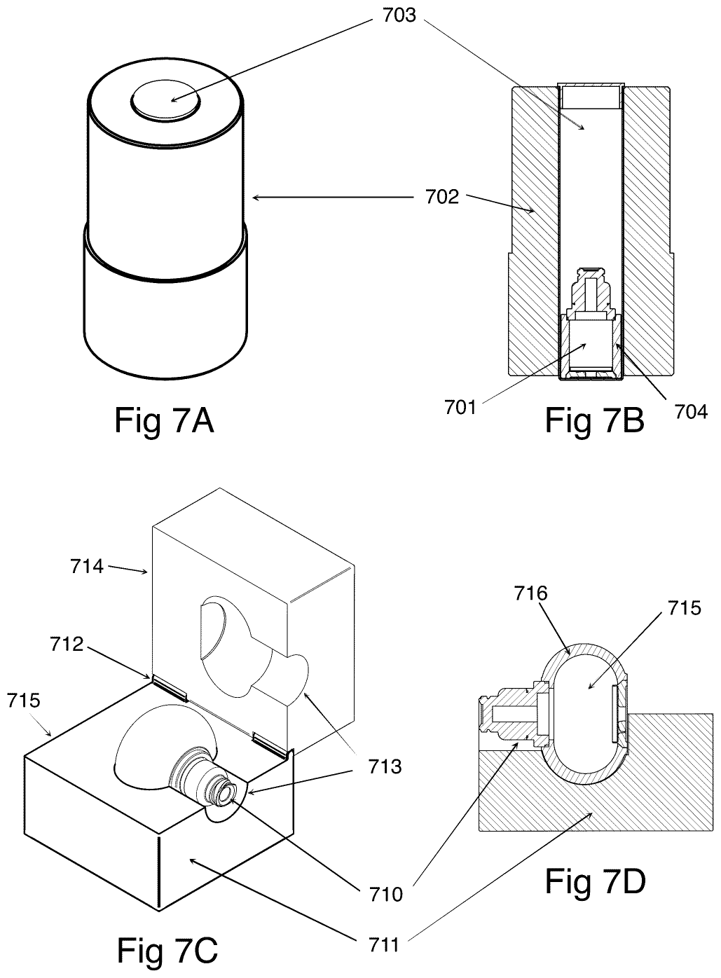

FIGS. 7A-7D show a pair of embodiments of quantitative vials that have different shapes. FIGS. 7A (oblique) and 7B (cross section) depict an embodiment where the vial (701) is measured in a detector (702) that has a pass-through well (703) with a cylindrical profile and openings at both ends. This would facilitate an automated counting process whereby vials were pushed through the well one after the other. The vial (701) that supports this embodiment has straight sides (704), as depicted in FIG. 7B), in contrast to the vial in FIGS. 1A-1F which has sides (112) that slope in towards the valve connection at top. Such a vial and detector configuration could be combined with a longer vial, allowing for larger sample size, as the vial could be positioned in the center of a longer detector without concern for settling effects. FIGS. 7C (oblique) and 7D (cross section) depict an embodiment where a vial (710) is measured in a detector (711) with a fold-over mechanism (712). The counting well (713) is in two parts, such that when the upper portion of the detector (714) is folded down to make contact with the lower portion (715), the sample in the vial will be counted efficiently as the detector surrounds the sample almost completely. Note that this fold-over design allows the sample volume (715) to be as large as desired without the constraint of fitting through an opening of a cylindrical well; in fact, the detector in this configuration does not require a circular opening at all. This design optimizes counting brightness for two reasons: less loss of activity through a relatively small or absent opening in the detector, and the possibility of larger sample size. This design allows for any shape of wall of the vial walls (716), shown here as roughly spherical.

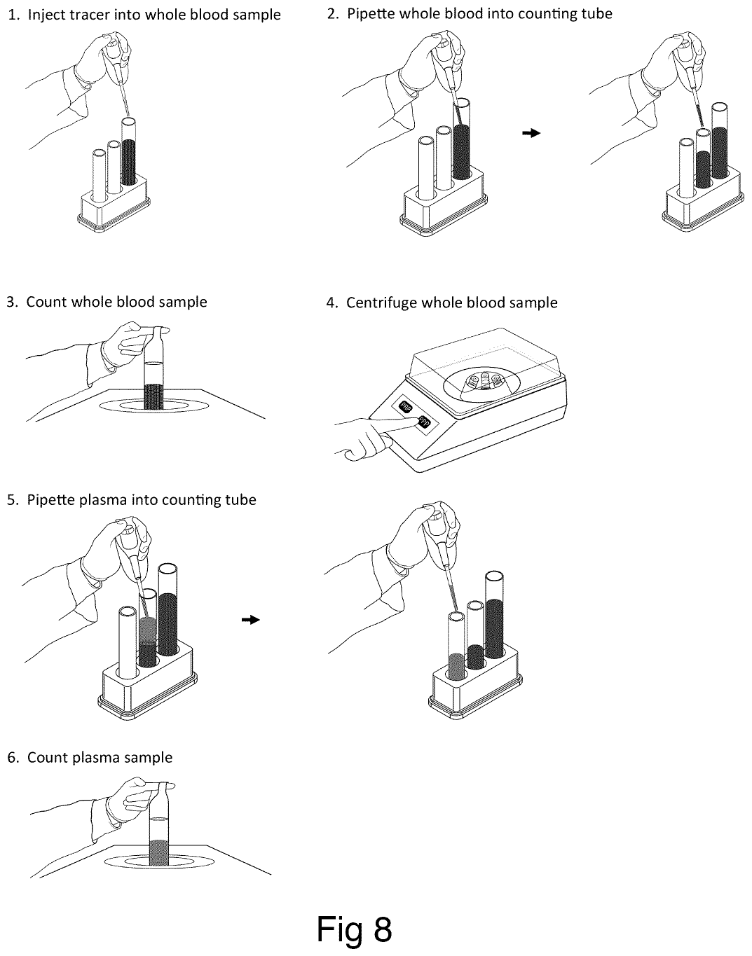

FIG. 8 shows one embodiment of a method of determining Hct using a tracer. In this embodiment, the tracer is added to the whole blood sample after it is collected from the patient, and precise equal volumes of blood and plasma are extracted by precision pipette and counted separately. This includes the steps of: 1) injecting tracer into a whole blood sample (shown here as injection into a tube of blood, but this tracer might be introduced into the blood of the subject via injection before the sample is collected); 2) pipetting a fixed quantity of blood into a counting tube; 3) counting the fixed quantity whole blood tube in a detector; 4) centrifuging whole blood from the sample with tracer to separate whole blood from plasma, which is accessible as the upper layer of fluid in the tube; 5) pipetting the same fixed quantity of plasma into another counting tube; 6) counting the plasma in the detector, and using the equation in section above to calculate the Hct.

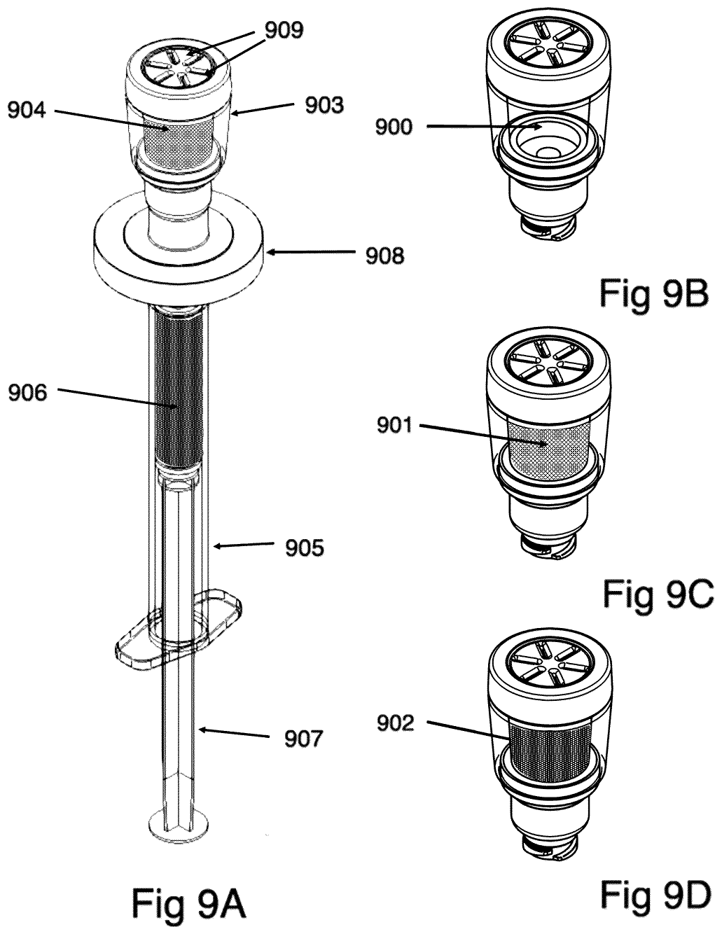

FIGS. 9A-9D illustrate an embodiment for a method of determining Hct with a tracer, using quantitative vials which can be filled to a precise volume with a syringe via luer-lock access, and a plasma separation chamber. This method has the advantage of not requiring any additional laboratory equipment, and can typically be performed by a non-laboratorian (i.e. a medical worker with phlebotomy but not laboratory skills). FIG. 9B shows a quantitative vial in an empty state, filled with air (900); FIGS. 9C and 9D show vials filled with plasma (901) and blood (902). FIG. 9A shows a method of filling a quantitative vial (903) with a precise amount of plasma (904), from a syringe (905) containing whole blood (906). As the syringe plunger (907) is pushed into the body of the syringe, the blood (906) is forced into a plasma separation chamber (908), which is connected to the syringe (905) below and the vial (903) above via luer-lock connections. The plasma separation chamber (908) is composed of a plasma separation membrane (i.e. a membrane that allows plasma to pass but impedes red cells) held across an opening; it has a sufficient area to be able to produce the desired quantity of blood to fill the vial without clogging—this is the reason for its larger diameter relative to the vial. Only plasma (904) passes through the plasma separation membrane; the vial (903) ends up completely filled with plasma (904), as the air in the vial (900) is displaced and exits through perforations in the vial (909). These perforations are behind a membrane which allows gases but not liquid to pass through.

What is claimed is:

1. A vial of precise volume, comprising an ingress with a standard fitting for accepting fluid and a valve to prevent backflow or outflow of the fluid, and an egress incorporating a membrane which passes air but retains fluid.

2. The vial of claim 1, wherein the standard fitting is a luer-lock mechanism.

3. The vial of claim 1, wherein the vial is formed from four separate components: an ingress adapter incorporating a luer lock and a valve, a membrane with a pressure resistance, defined as water entry pressure or WEP, sufficient to prevent flow of fluid, a lid to which the membrane is affixed, and a vial body which connects the lid to the adapter.

4. The vial of claim 3, wherein the lid supporting the membrane has a dense structure, increasing its effective WEP rating.

5. The vial of claim 3, wherein the lid supporting the membrane has a sparse structure, increasing the ease of pushing air out of the vial.

6. The vial of claim 1, wherein the vial body is formed from transparent material with sufficient clarity to allow visual inspection of the vial by a user, to verify that the vial is completely filled, with no visible air spaces.

7. The vial of claim 3, comprising smooth curved transitions between all components to ensure that no air is trapped in the vial while it is filled by eliminating spaces where bubbles could form and thereby prevent an exact desired volume from being achieved in the vial.

8. The vial of claim 1, wherein the vial is fitted with a second access point, equipped with a second adapter for accepting liquid via a luer lock or standard threaded connection and a second valve, to provide a means of applying or relieving pressure.

9. The vial of claim 1, wherein the inner surfaces of the vial are treated, either before or after assembly, with a finish coating that has the property of having low adhesion for blood.

10. The vial of claim 1, wherein the vial has a geometry that is matched with a geometry of a detector into which the vial is placed.

11. The vial of claim 10, wherein the vial fits within a cylindrical counting well that has a height relative to the depth of the counting well such that the entire sample in the vial is deep within the well.

12. The vial of claim 11, wherein the well is a complete cylindrical cutout, allowing vials to be inserted from one side and removed from the other.

13. The vial of claim 10, wherein the detector well has a fold-over design allowing the vial to be partially or completely encased in the detector.

14. The vial of claim 10, wherein a separate disposable sleeve is provided, of suitable size and dimensions to accept the vial, with the vial-enclosed sleeve fitting into the detector.

15. A kit for conducting an indicator dilution measurement, comprising a plurality of the vials of claim 1, and a reference solution of precise volume.

16. A method for determining the Hematocrit (Hct) of a subject, comprising the steps of:
a) introducing a tracer into the blood of the subject that marks the plasma component of the blood, and allowing sufficient time for said tracer to become equally distributed throughout the blood of the subject;
b) collecting a sample of the subject's blood;
c) measuring A_{wb}, the activity of the tracer in the whole blood sample from step (b);
d) extracting plasma from the whole blood sample from step (b);
e) measuring Aₚ, the activity of the tracer in the plasma sample from step (d); and
f) calculating the Hct of the subject using the equation
$$Hct = 1 - \frac{{A}_{wb}}{{A}_{p}} .$$

17. The method of claim 16, where a suitable tracer is already present and distributed in the subject's blood, so that the method is initiated at step (b).

18. The method of claim 16, where steps (a) and (b) are reversed in time, so that a whole blood sample is first collected from the subject; a tracer is introduced to the sample and distributed throughout the sample; and then steps (c) through (f) are performed.

19. The method of claim 16, where the tracer is a radioisotope, and the activity is measured in steps (c) and (e) using a radiation detector.

20. The method of claim 19, where the tracer is human serum albumin (HSA) labelled with I-131, I-125, or Tc-99m.

21. The method of claim 16, where the tracer is a fluorophore, and the activity is measured in steps (c) and (e) using a fluorescence detector.

22. A system for determining the Hematocrit (Hct) of a subject, comprising a tracer that marks the plasma component of the blood, a concentration counter capable of measuring the activity of said tracer, containers for presenting samples to the concentration counter, and a plasma separator capable of producing a sample of plasma from a sample of whole blood, using the method of claim 16.

23. The system of claim 22, wherein the containers for presenting samples to the concentration counter are vials that are filled to a precise volume, V_{wb} and Vₚ for whole blood and plasma, using an ordinary syringe, wherein the activities are calculated as
$${A}_{wb} = \frac{{Counts}_{wb}}{{V}_{wb}} and {A}_{p} = \frac{{Counts}_{p}}{{V}_{p}} ,$$
and wherein the vials are vials of precise volume, comprising an ingress with a standard fitting for accepting fluid and a valve to prevent backflow or outflow of the fluid, and an egress incorporating a membrane which passes air but retains fluid.

24. The system of claim 23, where the plasma separator is a chamber with an input port for whole blood, a plasma separating membrane, and an output port that connects to the ingress of the vials.

25. A system for automatically analyzing blood of a living subject at the point of care (POC), comprising a concentration counter configured to analyze one or more samples, a user interface operatively connected to the concentration counter and configured for entry and display of information, one or more processors operatively coupled to a memory and configured to execute programmed instructions stored in the memory to carry out a method comprising the steps of:
a) measuring a concentration of a tracer in a vial containing whole blood from the subject in the concentration counter to determine a background count of said tracer in the subject's blood;
b) recording the time at which the subject is injected with a precise, known volume of said tracer;
c) counting in the concentration counter blood drawn from the subject in one or more additional vials at one or more timed intervals after the injection;
d) determining the subject's Hct using plasma obtained from the whole blood samples of step (c), using the system of claim 22;
e) calculating, by the one or more processors, a blood volume (BV), plasma volume (PV), and red cell volume (RCV) for the subject using the subject's Hct;
f) calculating, by the one or more processors, an ideal blood volume (iBV), ideal plasma volume (iPV), and ideal red cell volume (IRCV) for the subject patient based on the subject's height, weight, and gender; and
g) displaying, by the one or more processors, at the user interface, the calculated volumes.
